(19) United States
(12) Patent Application Publication
(10) Pub. No.: US 2009/0198345 A1
(43) Pub. Date: Aug. 6, 2009

(54) CALCIUM SILICATE-BASED COMPOSITE CEMENT AND MANUFACTURING METHOD THEREOF

(75) Inventor: Shinn-Jyh Ding, Taichung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: CHUNG SHAN MEDICAL UNIVERSITY, Taichung City (TW)

(21) Appl. No.: 12/025,701

(22) Filed: Feb. 4, 2008

(51) Int. Cl. A61F 2/28

(52) U.S. Cl. 623/23.62

(57) ABSTRACT

The present invention provides a method for producing calcium silicate-based bone cement and a composition produced by the method. It further provides a novel mixture for bone tissue repair.

### FIELD OF THE INVENTION

This present invention relates to bone cement especially calcium silicate-based bone cements.

### BACKGROUND

Bone defects occur in a wide variety of clinical situations, and their reconstruction to provide mechanical integrity to the skeleton is a necessary step in the patient's rehabilitation. A direct chemical bond between the bone and the bone repair materials may be desirable. Several materials such as glass-ionomer cement, bioactive glass cement, and calcium phosphate cement that satisfy this requirement have attracted investigation as a suitable bone fixation/repair material due to their bioactivity and mechanical properties. Kokubo's group reported a bioactive bone cement composed of MgO—CaO—SiO₂—P₂O₅—CaF₂ glass powder and bisphenol-a-glycidyl methacrylate (Bis-GMA) resin (Tamura J, Kawanabe K, Kobayashi M, Nakamura T, Kokubo T, Yoshihara S, Shibuya T. Mechanical and biological properties of two types of bioactive bone cements containing: MgO—CaO—SiO₂—P₂O₅—CaF₂ glass and glass-ceramic powder. J Biomed Mater Res 1996; 30:85-94). Brown and Chow developed a calcium phosphate cement comprised of a mixture of particles of tetracalcium phosphate (Ca₄(PO₄)₂O) and dicalcium phosphate anhydrous (CaHPO₄), with which water mixed to form a paste that converted in situ to hydroxyapatite (Brown W E, Chow L C. Dental restorative cement pastes. U.S. Pat. No. 4,518,430, 1998).

In the 1970s, Carlisle reported that silicon (Si) was an important trace element in an early stage of bone formation as a result of earlier in vitro and in vivo studies (Carlisle E M, Silicon: a possible factor in bone calcification. Science 1970; 167:279-280). Silicon increases directly with calcium at relatively low calcium concentrations and falls below the detection limit at compositions approaching hydroxyapatite. The soluble form of Si may stimulate collagen type I synthesis and osteoblastic differentiation in human osteoblast-like cells (Reffitt D M, Ogston N. Jygdaohsingh R. Orthosilicic acid stimulates collagen type I synthesis and osteoblastic differentiation in human osteoblast-like cells in vitro. Bone 2003; 32:127-135). Klein et al. found that the more stable silica gels treated at 900 and 1000° C. showed some bone bonding, while the degraded gels sintering at the lower temperatures of 400 and 600° C. evoked a high cellular reaction of giant cells and lymphocytes (Klein C P A T, Pangjian Li, de Blieck-Hogervorst J M A, de Groot K. Effect of sintering temperature on silica gels and their bone bonding ability. Biomaterials 1995; 16:715-719). Several attempts have been made to prepare Si-containing bioactive materials such as bioactive glass and silicon-substituted hydroxyapatite (HA) to create new materials or improve the performance such as bioactivity and mechanical properties of bulk HA. Gibson et al. developed silicon-substituted hydroxyapatite by incorporating a small amount of silicon (0.4 wt %) into the structure of hydroxyapatite via an aqueous precipitation reaction (Gibson I R, Best S M, Bonfield W. Chemical characterization of silicon-substituted hydroxyapatite. J Biomed Mater Res 1999; 44:422-428). Silicate-based materials may be promising for use in the reconstruction of frontal sinus and spine, augmentation of craniofacial skeletal defects and osteoporosis, endodontics, and repair of periodontal bone defects.

The sol-gel-prepared materials are transformed to ceramics by heating at relatively low temperatures and have better chemical and structural homogeneity than those obtained by conventional glass melting or ceramic powder methods. It has been reported that materials prepared by a sol-gel process are more bioactive than the materials of the same compositions but prepared by other methods (Li P, de Groot K. Better bioactive ceramics through sol-gel process. J Sol-Gel Sci Technol 1994; 2:797-806). Certain hydroxyl groups such as Si—OH remaining in the sol-gel-prepared materials are assumed to promote hydroxyapatite formation by providing sites for calcium phosphate nucleation (Peltola T, Jokinen M, Rahiala H, Levänen E, Rosenholm J B, Kangasniemi I, Yli-Urpo A. Calcium phosphate formation on porous sol-gel-derived SiO₂ and CaO—P₂O₅—SiO₂ substrates in vitro. J Biomed Mater Res 1999; 44:12-21). Izquierdo-Barba et al. synthesized a bioactive glass with a composition of SiO₂ 80% and CaO 20% (in mol %) by sol-gel method and opened up new researches in the CaO—SiO₂ system (Izquierdo-Barba I, Salinas A J, Vallet-Regi M. In vitro calcium phosphate layer formation on sol-gel glasses of the CaO—SiO₂ system. J Biomed Mater Res 1999; 47:243-250). The same research group also found that the rate of apatite formation on the SiO₂—CaO glass with lower SiO₂ (50-70% in mol) is greater than those glasses with higher SiO₂ (80-90% in mol) when soaked in a simulated body fluid (Martínez A, Izquierdo-Barba I, Vallet-Regí M. Bioactivity of a CaO—SiO₂ binary glasses system. Chem Mater 2000; 12:3080-3088). In studies by Chang' group (Gou Z, Chang J. Synthesis and in vitro bioactivity of dicalcium silicate powders. J Eur Ceram Soc 2004; 24:93-99; Zhao W, Chang J. Sol-gel synthesis and in vitro bioactivity of tricalcium silicate powders. Mater Lett 2004; 58:2350-2353), sol-gel derived dicalcium silicate and tricalcium silicate powders could form a carbonate-containing hydroxyapatite layer on the surface when soaked in simulated body fluid. With the two calcium silicate powders, they can be mixed with water to produce a calcium silicate cement with the initial setting time of higher than 1 hour, on which the apatite precipitation needed to take several days in simulated body fluid (Zhao W. Wang J. Zhai W. Wang Z. Chang J. The self-setting properties and in vitro bioactivity of tricalcium silicate. Biomaterials 2005; 26:6113-6121; Gou Z. Chang J, Zhai W, Wang J. Study on the self-setting property and the in vitro bioactivity of β-Ca₂SiO₄. J Biomed Mater Res B: Appl Biomater 2005; 73B:244-251).

### SUMMARY OF THE INVENTION

This invention is to develop a sol-gel derived calcium silicate-based bone cement. This cement consists of calcium silicate biphasic powder as a solid phase and water or phosphate solution as a liquid phase.

This present invention provides a method for producing calcium silicate-based bone cement, comprising (a) mixing calcium salt with silicohydrides; (b) processing the mixture with a sol-gel process; (c) heating the mixture; (d) grinding the mixture into powder; and (e) adding the powder to water or phosphate solution.

The present invention further provides a mixture having the characteristics as follows: high bioactivity and fast setting after mixing with liquid phase of ammonium hydrogen phosphate solution.

### BRIEF DESCRIPTION OF THE DRAWINGS

The following detailed description, given by way of examples and not intended to limit the invention to the embodiments described herein, will best be understood in conjunction with the accompanying drawings, in which:

FIG. 1 illustrates XRD patterns for various as-sintered calcium silicate powders.

FIG. 2 illustrates DTS values of five different calcium silicate-based cements of which solid phases were sintered at a range of temperature. The ammonium hydrogen phosphate buffer solution was used as a liquid phase.

FIG. 3 illustrates surface SEM micrographs of equimolar Ca/Si powders sintered at 900° C. after being ground at different time.

FIG. 4 illustrates DTS values of calcium silicate cement with equimolar Ca/Si ratio of which the solid phase was ground at different time. The ammonium hydrogen phosphate buffer solution was used as a liquid phase.

FIG. 5 illustrates XRD patterns for various hardened cement specimens mixed with ammonium hydrogen phosphate buffer solution.

FIG. 6 illustrates surface SEM micrographs of the hardened cement specimens with equimolar Ca/Si before (A) and after (B) immersion for 1 hour in Hanks' solution. FIG. (C) is the cross-sectional micrograph of FIG. (B).

FIG. 7 illustrates DTS values of various cement specimens before and after immersion in Hanks' solution for predetermined periods of time. The ammonium hydrogen phosphate buffer solution was used as a liquid phase.

FIG. 8 illustrates XRD patterns for various cement specimens before and after immersion in Hanks' solution for predetermined periods of time. The ammonium hydrogen phosphate buffer solution was used as a liquid phase.

FIG. 9 illustrates SEM micrographs of U2OS cells cultured on cement specimens with equimolar Ca/Si ratio at various incubation periods. The liquid phase of the cement was ammonium hydrogen phosphate buffer solution.

FIG. 10 illustrates MTT assay for proliferation of U2OS cultured in the presence of the cement specimens with equimolar Ca/Si ratio at various incubation periods. The liquid phase of the cement was ammonium hydrogen phosphate buffer solution.

### DETAILED DESCRIPTION OF THE INVENTION

The present invention provides a method for producing calcium silicate-based bone cement, comprising the following steps:

(a) mixing calcium salt with silicohydrides;

(b) processing the mixture with a sol-gel process;

(c) heating the mixture;

(d) grinding the mixture into powder; and

(e) adding the powder to water or phosphate solution.

In a preferred embodiment, the calcium salt is calcium nitrate and the silicohydride of said method has the following formula:

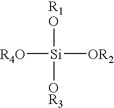

wherein R₁, R₂, R₃ and R₄ are C₁₋₆ alkyl.

In a more preferred embodiment, the silicohyride of said method has the following formula:

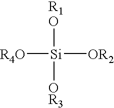

wherein R₁, R₂, R₃ and R₄ are C₂H₅.

In a preferred embodiment, said mixture has a molar ratio of calcium to silicon ranges from 10 to 0.1. In a more preferred embodiment, the mixture has the molar ratio of calcium to silicon range from 4 to 0.25.

The sol-gel process of said method comprises the following steps:
(a) mixing the mixture calcium and silicon precursors with ethanol and/or dilute nitric acid for 1˜12 hours;
(b) placing the mixture at the temperature of 20˜80° C. for 1˜7 days; and
(c) drying the mixture at 100˜150° C.

The heating process of said method comprises the following steps:
(a) heating the mixture to 700˜1300° C. at the rate of 1˜40° C./min;
(b) maintaining the mixture at a constant temperature ranges from 700˜1300° C.; and
(c) cooling the mixture to room temperature by air-cooling, water-cooling or fast cooling techniques to obtain calcium silicate powder.

The grinding process comprises the following steps:

(a) mixing the powder with alcohol;

(b) grinding the powder with a ball miller for 0.5˜3 days; and

(c) drying the powder at 100˜150° C.

In a preferred embodiment, the particle size of the powder ranges from 0.01 to 50 micrometers. In a more preferred embodiment, the particle size of the powder ranges from 0.1 to 5 micrometers.

In a preferred embodiment, the powder is added to water for 10˜60 seconds and the ratio of water to powder is 0.3-2 mL/g. In a more preferred embodiment, the ratio of water to powder is 0.4-0.7 mL/g.

In a preferred embodiment, the powder is added to phosphate solution for 10˜60 seconds and the ratio of phosphate solution to powder is 0.4-2 mL/g. In the best embodiment, the ratio of phosphate solution to powder is 0.5-0.8 mL/g.

In a preferred embodiment, the anion of the phosphate solution is phosphate radical (PO₄³⁻), monohydric phosphate radical (HPO₄²⁻) or dihydric phosphate radical (H₂PO₄⁻) with a concentration of 0.12˜5 M, and the cation of the phosphate solution is ammonium, or a member of group I A. Preferably, the cation of the phosphate solution is ammonium, sodium or potassium.

The present invention further provides a mixture comprising calcium silicate-based bone cements having the following characteristics:

(a) the diametral tensile strength values of the cement range from 0.9 to 2.9 MPa; and

(b) the setting times of the cement are in the range of 3 to 20 minutes.

Furthermore, the mixture can be applied to orthopedic surgery, spine fusion surgery or dental application. It can also serve as replacement bone or tooth material. When applied to human, it can be orally delivered with an excipient.

### EXAMPLE

The example below is non-limiting and is merely representative of various aspects and features of the present invention.

### Example 1

### Phase Composition of Calcium Silicate Powders

Tetraethyl orthosilicate (Si(OC₂H₅)₄, TEOS) and calcium nitrate (Ca(NO₃)₂·4H₂O) were used as precursors for SiO₂ and CaO, respectively, and nitric acid as catalyst. Ethanol was used as the solvent. The molar ratio of SiO₂/CaO is in the range of 7/3 to 3/7, as listed in Table 1. Various calcium silicate powders were synthesized by sol-gel method. The general procedure of sol-gel route, such as hydrolysis and ageing, was adopted. Briefly, the TEOS was hydrolyzed with the sequential addition of 2 N HNO₃ and absolute ethanol for 1 hour stirring separately. The required amount of Ca(NO₃)₂·4H₂O was added to the above ethanol solution, the mixed solutions were stirred for an another 1 hour. The molar ratio of (HNO₃+H₂O):TEOS:ethanol was 10:1:10. The sol solution was sealed and aged at 60° C. for 1 day.

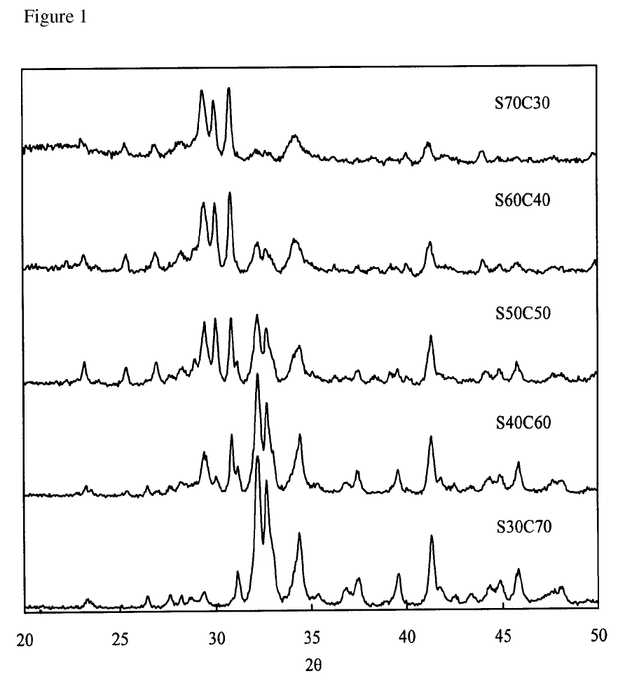

After solvent vaporization of the above-mentioned mixture solution in an oven at 120° C., the as-dried gel was heated in air to 900° C. for holding 1 hour, and then cooled to room temperature to produce various calcium silicate powders. Phase analysis was performed using Shimadzu XD-D1 X-ray diffractometer (XRD) with Ni-filtered Cukα radiation operated at 30 kV and 30 mA at a scanning speed of 1°/min. The XRD spectra of the various as-prepared CaO—SiO₂ powders are presented in FIG. 1. The diffraction maximum between 29 and 35° at 2θ can be attributed to different crystalline phases of calcium silicates, such as wollastonite and dicalcium silicate. A small amount of CaO at 2θ=37.5° appeared for the powders with silicon content lower than calcium such as S50C50, S40C60 and S30C70 specimens.

TABLE 1
Composition, setting time and diametral tensile strength
of SiO₂—CaO system cements after mixing with
water or ammonium hydrogen phosphate solution

| | | | | Ammonium hydrogen | |
|---|---|---|---|---|---|
| | | Water | | phosphate | |
| | Composition | Setting | | Setting | |
| | (molar ratio) | time | | time | |
| Sample | SiO₂:CaO | (min) | DTS (MPa) | (min) | DTS (MPa) |
| S70C30 | 7:3 | 76.3 ± 3.5 | 0.2 ± 0.0 | 9 ± 0.5 | 2.0 ± 0.2 |
| S60C40 | 6:4 | 28.6 ± 2.1 | 2.0 ± 0.3 | 7 ± 0.7 | 2.3 ± 0.1 |
| S50C50 | 5:5 | 30.0 ± 1.0 | 2.3 ± 0.2 | 5 ± 0.6 | 2.9 ± 0.2 |
| S40C60 | 4:6 | 21.7 ± 1.5 | 1.8 ± 0.4 | 4 ± 0.4 | 1.1 ± 0.1 |
| S30C70 | 3:7 | 10.0 ± 1.0 | 0.8 ± 0.2 | 3 ± 0.5 | 0.9 ± 0.1 |

### Example 2

### Setting Time and Diametral Tensile Strength of the Cement

The powders sintered at 900° C. for 1 hour were ball-milled using agate jars with agate grinding media in an ethanol medium for 24 hours under Retsch centrifugal ball mill S 100. After drying, in order to prepare the cement, 0.2 g powder was mixed water or the (NH₄)₂HPO₄—NH₄H₂PO₄ buffer solution (pH 7.4) using a liquid-to-powder (L/P) ratio of 0.5-0.7 mL/g depending on the kind of the cement. The setting time of the cements was tested using the 400-g Gillmore needle with a diameter of 1 mm according to the international standard ISO 9917-1 for water-based cements (ISO 9917-1, Dentistry-water-based cements part1: powder/liquid acid-base cements. International Standard Organization, 2003). The setting time was recorded when the needle failed to create an indentation of 1 mm in depth in three separate areas. After mixing the cement specimens were placed into a cylindrical stainless steel mould to form the specimen dimension of 6 mm (diameter)×3 mm (height), and stored in an incubator at 100% relative humidity and 37° C. for 1 day. The diametral tensile testing of cement specimens was conducted on an EZ-Test machine (Shimadzu, Kyoto, Japan) at a loading rate of 0.5 mm/min. The diametral tensile strength (DTS) value of the cement specimens was calculated from the relationship DTS=2P/πbw, where P is the peak load (Newton), b is the diameter (mm) and w is the thickness (mm) of the specimen. The maximal compression load at failure was obtained from the recorded load-deflection curves. At least ten specimens from each group were tested. Table 1 presents the results of setting times and DTS values of five calcium silicate cement specimens. When mixed with water, the setting time ranged from 10 to 76 minutes, significantly depending on the used calcium silicate powders. With the increase in the concentration of calcium component, the setting time of the cement became shorter. In contrast, all cement specimens hardened within 10 minutes after mixing with ammonium hydrogen phosphate solution. Regarding the DTS, the cement specimens with the greatest SiO₂ amount had a value of 2.0 MPa after mixing with ammonium hydrogen phosphate solution, whereas the greatest calcium amounts of cement specimens became 0.9 MPa. The highest DTS value in the Ca—Si cement system was 2.9 MPa that belonged to the equimolar Ca/Si ratio cement mixed with ammonium hydrogen phosphate solution.

### Example 3

### Effect of Sintering Temperature and Nitric Acid on Diametral Tensile Strength of the Cement

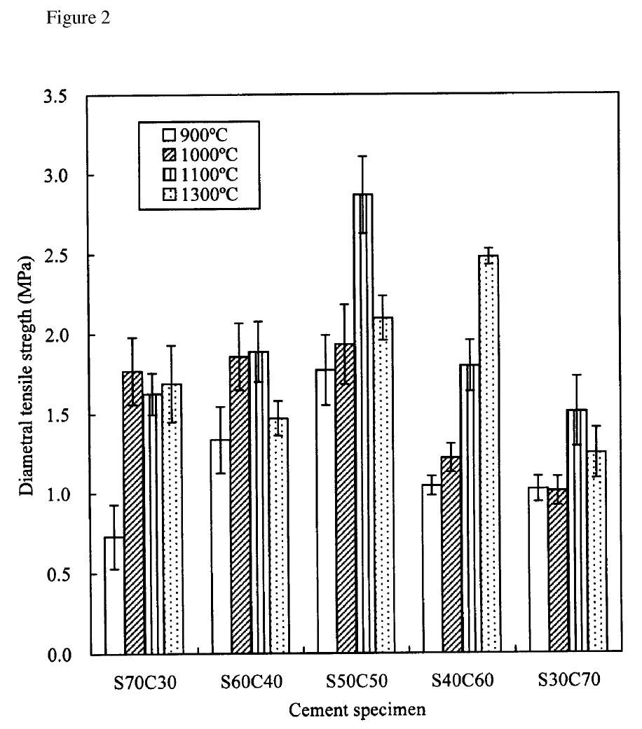

In the case of sintering temperature, the as-dried gel was heated in air to 900, 1000, 1100 and 1300° C. at a heating rate of 10° C./min for holding 1 hour. Sequentially various as-sintered powders were ball-milled using agate jars with agate grinding media for 1 hour under Retsch centrifugal ball mill S 100. After drying in oven, the powders was mixed with the (NH₄)₂HPO₄—NH₄H₂PO₄ solution to measure DTS. FIG. 2 shows the effect of various sintering temperatures on DTS for five different calcium silicate materials. It can be noted that the lowest DTS value was measured for specimens sintered at 900° C., whereas the maximum value was dependent on the type of the specimens obtained at different temperature. The general trend was that the DTS values increased from 900 to 1100° C. To further understanding processing parameters of a sol-gel route, the effect of nitric acid as catalyst on DTS was studied. The amount of nitric acid was replaced by pure water during sol-gel processing. The sintering temperature was 900° C. and the grinding time was 24 hours. The Ca/Si=5/5 cement prepared without nitric acid had a lower strength of 2.2±0.2 MPa than that of 2.9±0.2 MPa obtained for the cement with the addition of nitric acid.

### Example 4

### Effect of Grinding Time on Diametral Tensile Strength and Morphology of the Cement

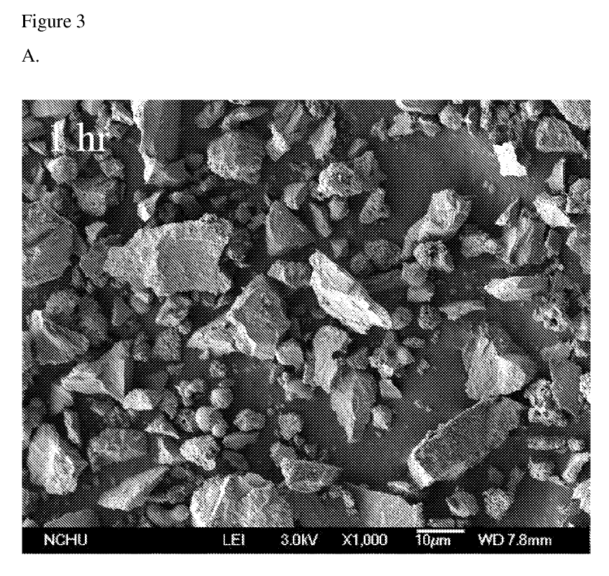

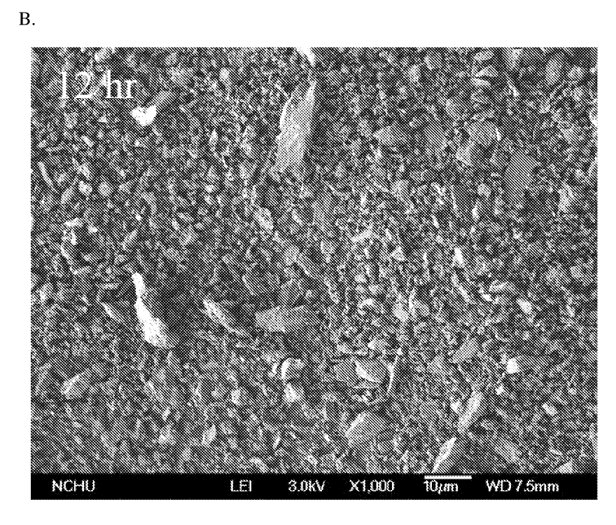

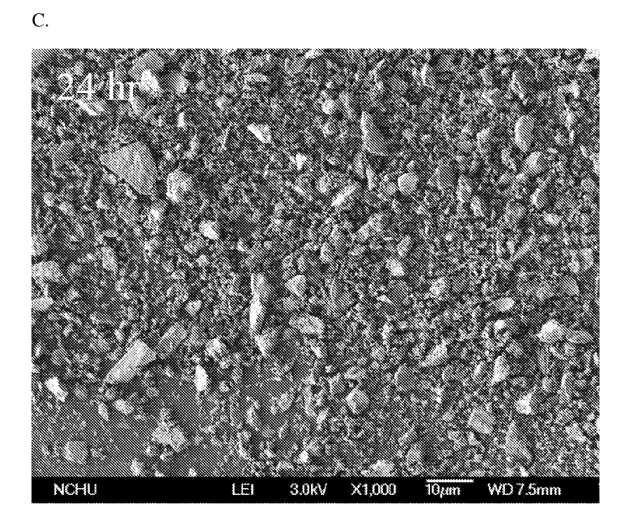

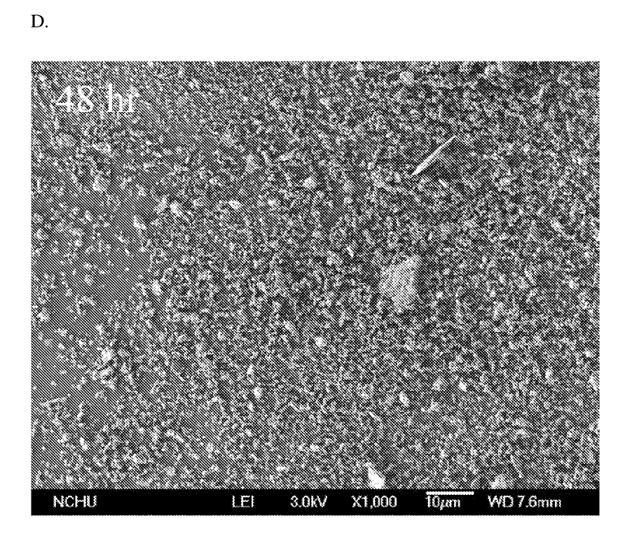

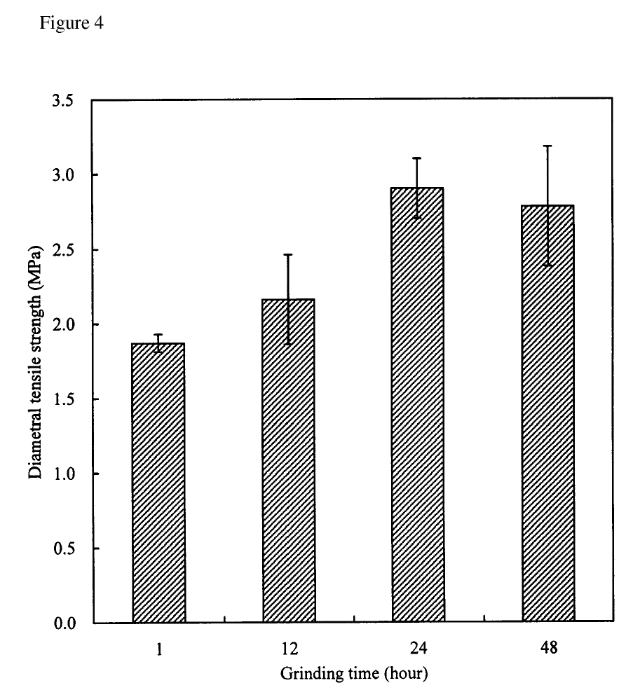

As for the effect of grind time, four different periods of time were performed for Ca/Si=5/5 powders sintered at 900° C. The powder morphology was studied as a function of milling time by JEOL JSM-6700F field emission scanning electron microscope (SEM). For 1 hour of grinding, the milled powders were formed of small irregular particles, ranging from 1 to 20 μm (FIG. 3). As a general trend, no spectacular changes in morphology were observable for grinding time higher than 12 hours, except there was a slight tendency to fine down to less than 1 μm. The relation of grinding time and DTS of the cement specimens was also evaluated. The ammonium hydrogen phosphate buffer solution was used as a liquid phase. The DTS values of the cements increased with increasing grinding time up to the optimum time of 24 hours (FIG. 4).

### Example 5

### Phase Composition of the Cement

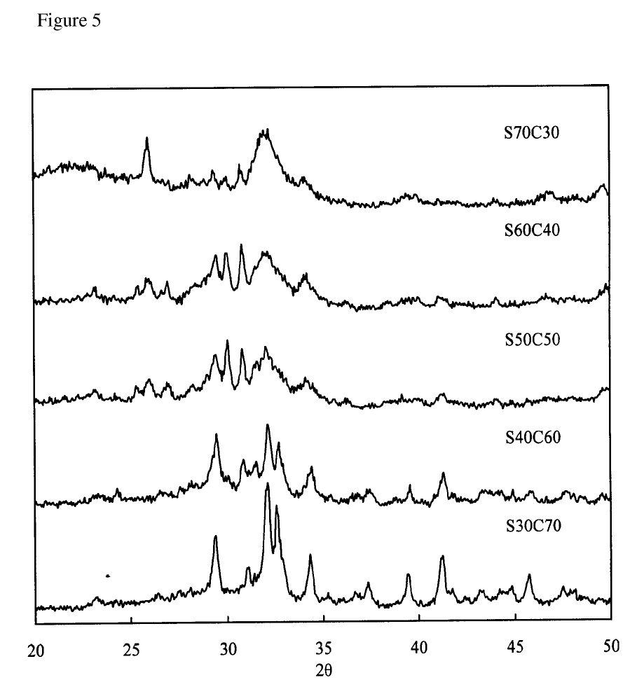

XRD was used to analyze the phase composition of the hardened cement specimens. When the liquid phase of ammonium hydrogen phosphate was added to the calcium silicate powders, the product of the hydration process was apatite in combination with a calcium silicate hydrate (CaO—SiO₂—H₂O, C—S—H) gel, as shown in FIG. 5.

### Example 6

### Morphology of the Cement after Immersion in Physiological Solution

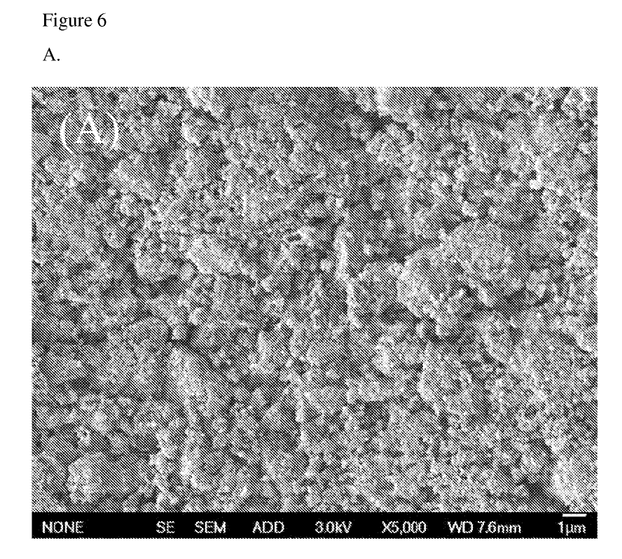

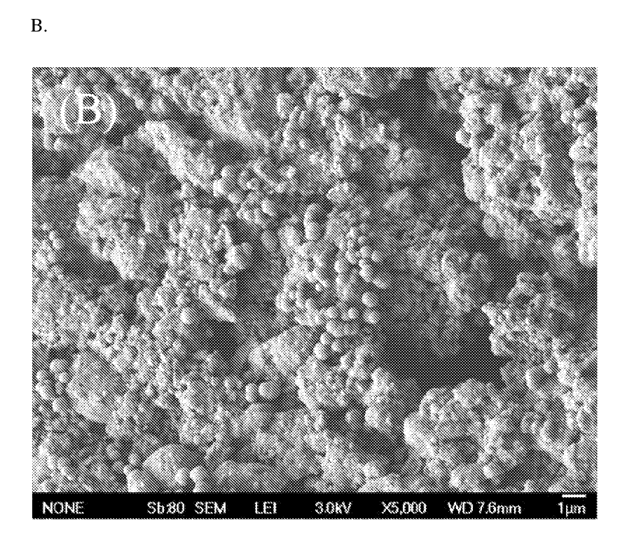

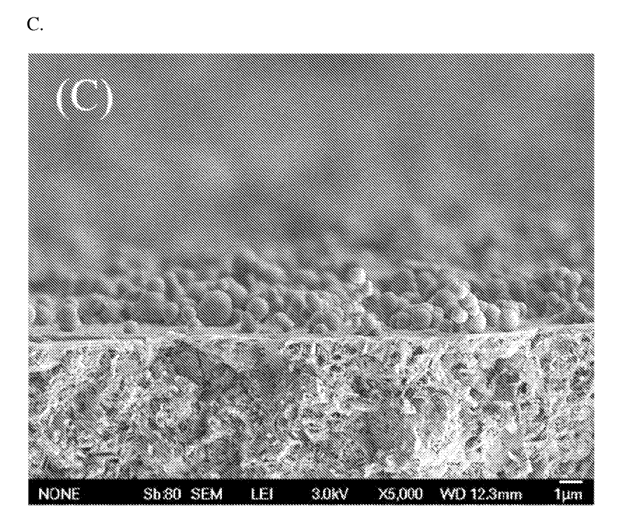

After mixing with ammonium hydrogen phosphate solution, each hardened specimen that was stored in an incubator at 100% relative humidity and 37° C. for one day was immersed in physiological solution for the predetermined periods of time at 37° C. to evaluate the cement bioactivity. The extracellular Hank's solution (Pourbaix M. Electrochemical corrosion of metallic biomaterials. Biomaterials 1984; 5:122-134) with an ionic composition similar to that of human blood plasma was used as immersion solution. This solution consisted of 8.00 g NaCl, 0.35 g NaHCO₃, 0.40 g KCl, 0.06 g KH₂PO₄, 0.10 g MgCl₂·6H₂O, 0.14 g CaCl₂, 0.06 g Na₂HPO₄·2H₂O, 0.06 g MgSO₄·7H₂O, 1.00 g glucose in 1000 ml distilled H₂O and had an initial pH of 7.4. The solution in a shaker water bath was not changed daily. After immersion, the specimens were removed from the vials to observe morphologies using a SEM. FIG. 6 indicates that the present cement with equimolar Ca/Si ratio induced the formation of apatite spherulites, indicating the bioactivity, when immersed in physiological solution as short as 1 hour.

### Example 7

### Diametral Tensile Strength for Various Immersed Cements

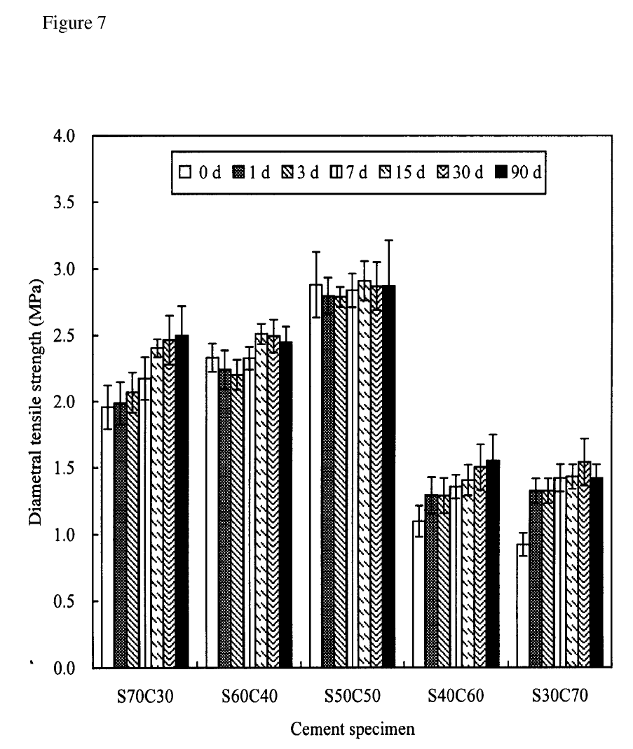

After immersion in Hanks' solution for different periods of time, the specimens were removed from the vials and performed the tensile testing using an EZ-Test machine. At least ten specimens from each group were tested. Essentially the DTS of all immersed cement specimens did not decrease with increasing immersion time (FIG. 7).

### Example 8

### Phase Composition of Immersed Cements

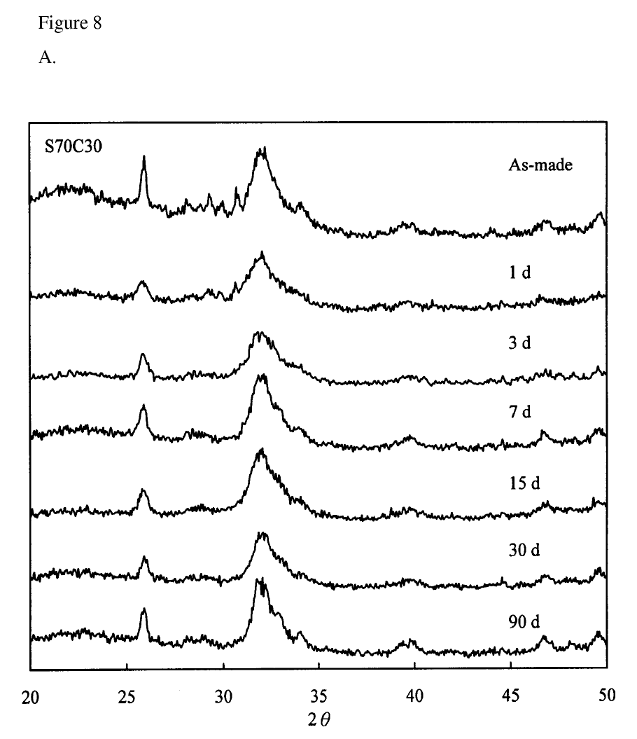

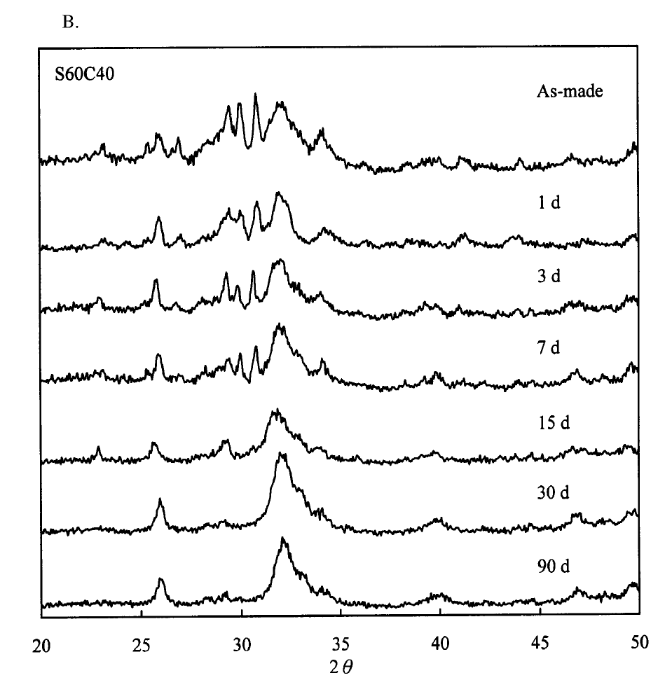

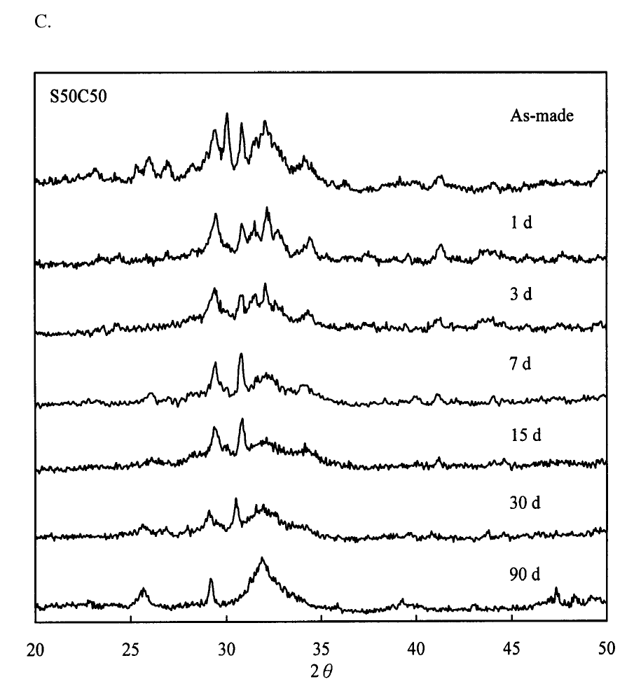

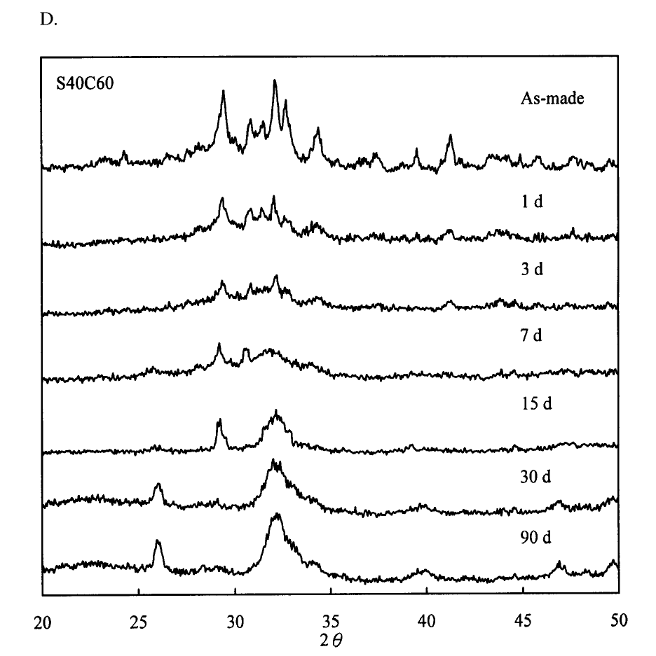

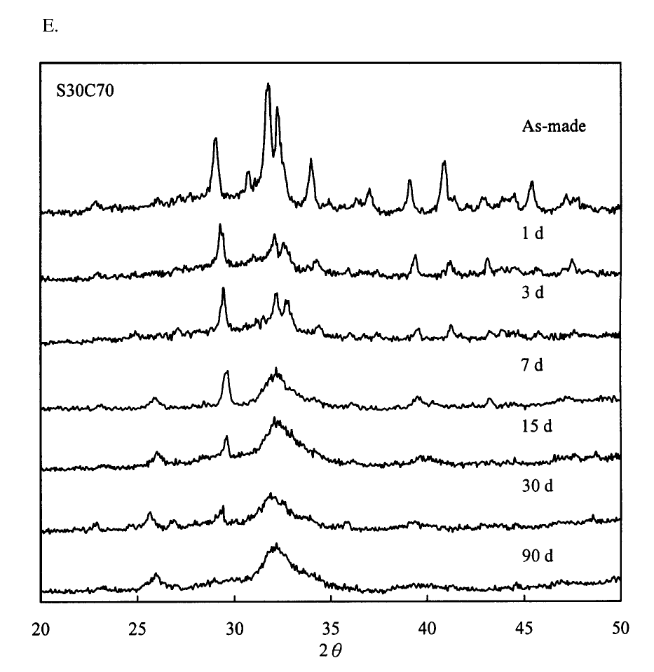

After immersion in Hanks' solution for a series of time, the specimens were removed from the vials. The phase evolution of the immersed cement specimens was carried out using an XRD. The intensities of the predominant phases with sharp peaks at 2θ=30-35° and the calcium silicate hydrate in all as-made cement decreased with increasing immersion time when immersed in simulated physiological solution (FIG. 8). The broadening of major apatite peaks roughly between 31 and 34°(2θ) indicated an immersion-induced precipitation of an amorphous apatite phase. The XRD results depicted that the five calcium silicate cements had a good bioactivity when soaked in simulated physiological solution.

### Example 9

### Morphology of Cell

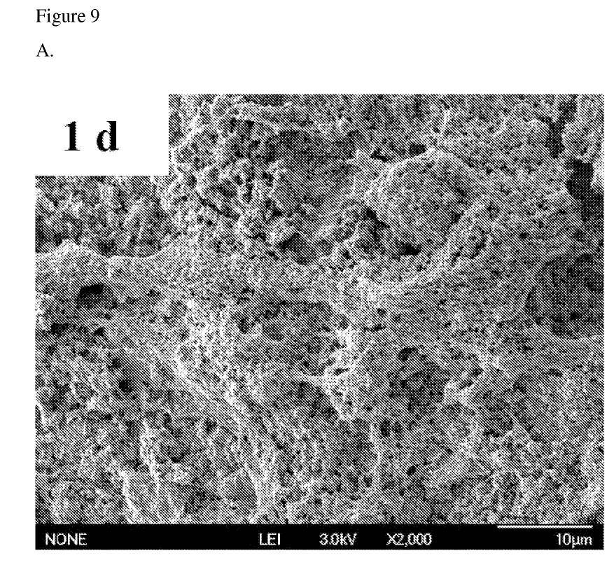

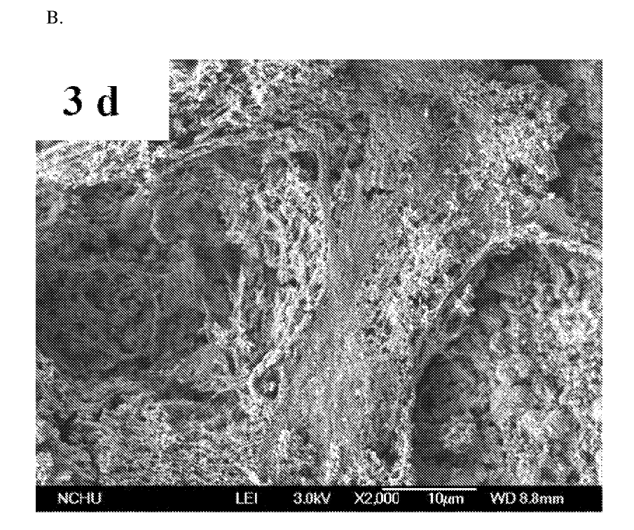

Cement biocompatibility was evaluated by incubation with human osteosarcoma cell line U2OS (ATCC, HTB 96, Manassas, Va., USA). Sample-free cultures were used as controls. For cell culture, the 1-day setting cement discs were sterilized by soaking in 75% ethanol and exposure to ultraviolet (UV) light for 2 hour. Single cell suspensions of U2OS were seeded into wells of a 24 well plate containing test specimen at 1×10⁴ cells per well and grown in McCoy's medium (Sigma) supplemented with 10% fetal bovine serum (FBS) and 1% antibiotics-antimycotic. Cultures were incubated at 37° C. in a 5% CO₂ atmosphere for 1, 3 and 7 days. After incubation, specimens were washed by phosphate buffer solution three times and fixed with 2% glutaraldehyde for 3 hour. Then the cements were dehydrated in a graded ethanol series for 20 minutes at each concentration and dried by liquid CO₂ using a critical point dryer device (LADD 28000, LADD, Williston, Vt.). The dried specimens were mounted on stubs, coated with gold layer. The cell morphology on the cement surface was observed using JEOL JSM-6700F SEM. The evaluation of SEM images confirmed that cells appeared to have firmly anchored on the cement surfaces that had an equimolar Ca/Si ratio (FIG. 9).

### Example 10

### Evaluation of Cell Viability

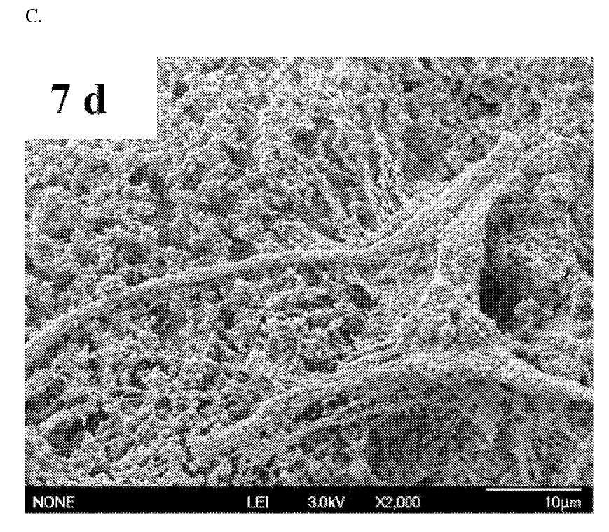

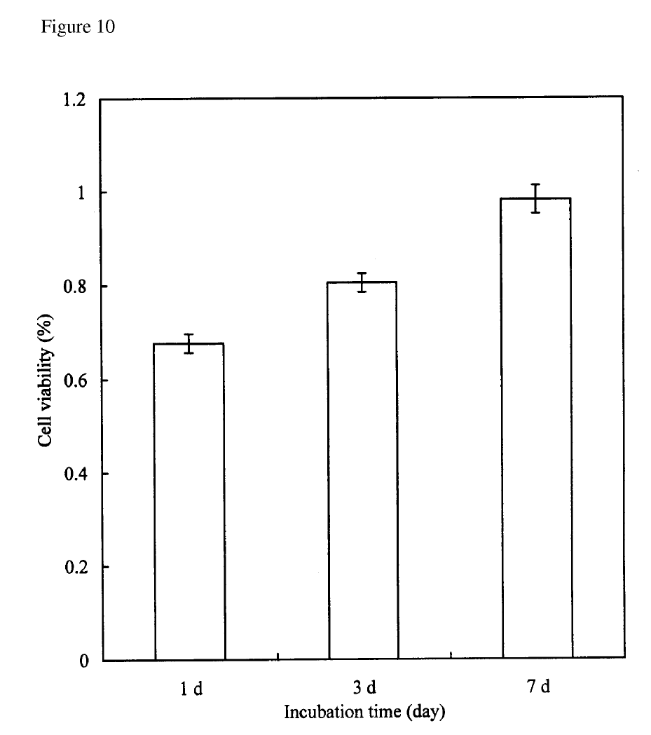

To quantitatively evaluate cell viability, MTT (3-(4,5-dimethylthiazol-2-yl)-2,5-diphenyltetrazolium bromide) in vitro toxicology assays were performed by adding reconstituted 2 mL MTT to the each U2OS cell well containing test cement specimen. After incubation for 4 hours, 2 mL of DMSO was also added to each well. The plates were then shaken until the purple crystals had dissolved, and the solution in each well was transferred to a 96-well tissue culture plate. The absorbance at 570 nm was read with a Σ960 ELISA microplate reader (Metertech Inc, Taipei, Taiwan). Cell viability (%) was obtained by dividing the value of the cement specimen by that of the control one without cement specimen. In FIG. 10 MTT assay shows that the number of viable cells increased with an increased incubation, indicating a good biocompatibility.

What is claimed is:

1. A method for producing calcium silicate-based bone cement, comprising
(a) mixing calcium salt with silicohydrides;
(b) processing the mixture with a sol-gel process;
(c) heating the mixture;
(d) grinding the mixture into powder; and
(e) adding the powder to water or phosphate solution.

2. The method of claim 1, wherein the calcium salt is calcium nitrate.

3. The method of claim 1, wherein the silicohydride has the following formula: 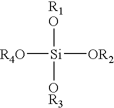
wherein R₁, R₂, R₃ and R₄ are C₁₋₆ alkyl.

4. The method of claim 1, wherein R₁, R₂, R₃ and R₄ are C₂H₅.

5. The method of claim 1 wherein mixture has a molar ratio of calcium to silicon ranges from 10 to 0.1.

6. The method of claim 5, wherein mixture has a molar ratio of calcium to silicon ranges from 4 to 0.25.

7. The method of claim 1, wherein the sol-gel process comprises following steps:
(a) mixing the mixture of claim 1 with ethanol and/or dilute nitric acid for 1˜12 hours;
(b) placing the mixture at the temperature of 20˜80° C. for 1˜7 days; and
(c) drying the mixture at 100˜150° C.

8. The method of claim 1, wherein the heating process comprises following steps:
(a) heating the mixture to 700˜1300° C. at the rate of 1˜40° C./min;
(b) maintaining the mixture at a constant temperature ranges from 700˜1300° C.; and
(c) cooling the mixture to room temperature by air-cooling, water-cooling or fast cooling techniques to obtain calcium silicate powder.

9. The method of claim 1, wherein the grinding process comprises following steps:
(a) mixing the powder with alcohol;
(b) grinding the powder with a ball bill for 0.5˜3 days; and
(c) drying the powder at 100˜150° C.

10. The method of claim 9, wherein the, the particle size of the powder ranges from 0.01 to 50 micrometers.

11. The method of claim 10, wherein the particle size of the powder ranges from 0.1 to 5 micrometers.

12. The method of claim 1, wherein the powder is added to water for 10˜60 seconds.

13. The method of claim 1, wherein the ratio of water to powder is 0.3-2 mL/lg.

14. The method of claim 13, wherein the ratio of water to powder is 0.4˜0.7 mL/lg.

15. The method of claim 1, wherein the powder is added to phosphate solution for 10˜60 seconds.

16. The method of claim 1, wherein the ratio of phosphate solution to powder is 0.4-2 mL/lg.

17. The method of claim 1, wherein the ratio of phosphate solution to powder is 0.5˜0.8 mL/lg.

18. The method of claim 1, wherein the anion of the phosphate solution is phosphate radical (PO₄³⁻), monohydric phosphate radical (HPO₄²⁻) or dihydric phosphate radical (H₂PO₄₋).

19. The method of claim 1, wherein the cation of the phosphate solution is ammonium or a member of group I A.

20. The method of claim 1, wherein the cation of the phosphate solution is ammonium, sodium or potassium.

21. The method of claim 18, wherein the concentration of anion is 0.12˜5 M.

22. A mixture comprising calcium silicate-based bone cements having the following characteristics:
(a) the diametral tensile strength values of the cement range from 0.9 to 2.9 MPa; and
(b) the setting times of the cement are in the range of 3 to 20 minutes.

23. The mixture of claim 22, which is applied to orthopedic surgery, medical or dental application.

24. The mixture of claim 22, which is applied to replacement bone or tooth material.

25. The mixture of claim 22, which is orally delivered with an excipient.
